(19) United States
(12) Patent Application Publication
(10) Pub. No.: US 2025/0195152 A1
(43) Pub. Date: Jun. 19, 2025

(54) ACCESS SYSTEMS AND METHODS

(71) Applicant: X9, Inc., Wallingford, CT (US)

(72) Inventors: Joshua Makower, Los Altos Hills, CA (US); Earl Bright II, Sunnyvale, CA (US); John Chang, Los Altos, CA (US); Eric Taylor, West Harford, CT (US); Jonathan Podmore, San Carlos, CA (US); Emma Essock-Burns, San Francisco, CA (US); John Hanley, Manhattan Beaxh, CA (US)

(21) Appl. No.: 19/069,968

(22) Filed: Mar. 4, 2025

Related U.S. Application Data
(63) Continuation of application No. PCT/US23/32009, filed on Sep. 5, 2023.
(60) Provisional application No. 63/404,010, filed on Sep. 6, 2022.

(51) Int. Cl. A61B 34/20; A61M 1/30

(52) U.S. Cl. CPC A61B 34/20; A61M 1/301; A61B 2034/2055

(57) ABSTRACT

Systems and methods for dialysis cannulation including an apparatus that includes or a method involving, structure configured for placing a needle or cannula to provide vascular access. In one approach, a self-cannulation system is provided for hemodialysis. In one aspect, the system guides movement of the needle through an acceptable access path and prevents movement of the needle through non-acceptable paths or insertion of the needle beyond a defined terminal point.

### FIELD OF THE DISCLOSURE

The present disclosure generally relates to systems and methods to cannulate vasculature or grafts.

### BACKGROUND OF THE DISCLOSURE

There is a continuing need for an effective alternative approach to providing vascular access and in particular for hemodialysis. Moreover, there is a need for an access system for clinic or home use which is easy and effective to use.

Hemodialysis has become a conventional approach to treat individuals with kidney disease. A hemodialysis procedure, commonly referred to as dialysis, involves filtering wastes from a person's blood and thus supports or replaces biological functions provided by a healthy kidney. Effective dialysis results in facilitating balancing substances residing in blood including calcium, potassium and sodium, and also can aid in controlling blood pressure.

Hemodialysis involves transporting blood through a dialyzer which functions as a filter to balance blood constituents, and acts much like an external kidney. In a hospital or clinic setting, a medical practitioner is tasked with setting up the dialyzer and hooking a patient up to the dialyzer. This involves gaining access to and placing needles connected or connectable to a cannula within the patient's vasculature and connecting tubes to and from the dialyzer.

Typically, a hemodialysis treatment lasts about four hours and is conducted three times per week, but more frequent and longer treatments can be necessary for some patients. Adding to this, the time to travel to a treatment center and waiting to be hooked up and disconnected from a dialyzer of course adds up to a very significant commitment from the patient both in time and effort.

It is due to these limitations associated with dialysis performed at treatment centers that more efficient hemodialysis including cannulation has become a desirable alternative either in the treatment center or at home. It is thus desirable to make dialysis as well as assessment of insertion sites and cannulation itself more efficient and/or require less skill. Studies have shown that home dialysis five to seven times a week has dramatically better outcomes in many ways including a longer life and better survival. When dialysis is performed at home, there is no need to travel to a dialysis center. There is also more flexibility in home dialysis as the patient is able to choose a convenient time for dialysis and has a greater sense of control from being independent and doing treatments themselves.

However, unless in-home care is engaged, there is most often no medical professional in the home setting to monitor treatments or answer immediate questions. Also, significantly, in the treatment center health care professionals are less available to assist with or perform the cannulation techniques required to insert needles within patient vessels so that the patient can be hooked up to a dialyzer. Cannulation is a skilled nursing task. Also, self-cannulation can be a daunting task for many patients and many patients can lack the necessary dexterity or skill to repeatably, effectively and efficiently insert needles to gain access to vasculature through insertion sites. Since proper repeatable cannulation is so critical to successful hemodialysis and the avoidance of infection and other complications, unassisted home dialysis is not currently a practical alternative to large populations of patients.

Accordingly, there is a need for effective and efficient devices and approaches to cannulation and for minimizing or reducing the time involved in receiving hemodialysis, as well as for doing so in a treatment center or home setting with simple-to-use systems that help to minimize trauma and infection. These approaches should be associated with predictable results and be relatively easy to employ.

The present disclosure addresses these and other needs.

### SUMMARY OF THE DISCLOSURE

Briefly and in general terms, the present disclosure is directed towards access systems and methods involving a cannulation system that facilitates providing vascular access. Such approaches to the cannulation system are configured to define an acceptable access path that prevents a user or a system from entering non-acceptable paths or insertion beyond a defined terminal point, as well as a small range of acceptable paths and a most desired path. In one aspect, the defined terminal point resides within walls defining the target graft or vessel.

The cannulation system is configured for use in center or in home dialysis and can be operated by the patient, skilled healthcare workers or unskilled personnel. The cannulation system, in one embodiment, includes a detection bed, an armature attached to the detection bed and a needle retaining fixture attached to the armature. The detection bed is provided with structure that accomplishes repeatably and consistently affixing a patient's body part to the detection bed and relative to the armature. The cannulation system is particularly suited for the patient that requires multiple, repeated cannulations. Due to the pre-planning and pre-determination of cannulation sites and pathways to the sites, the system is thus set up for enabling quick, repeatable, identical arm/vessel placement and confirmation, without having to gather information about the patient or cannulation sites with each cannulation procedure.

In one embodiment, one or more fiducials are implanted in the patient and through a pre-scanning process, the fiducials are used to register a patient and to map a precise location of targeted vasculature whether it be an artificial graft or natural access anatomy including fistulas or vessels themselves. Additional scanning is performed as needed such as on a periodic basis so that the position of the targeted vasculature or graft is confirmed relative to implanted fiducial(s) or other landmarks. In one approach, additional scanning can be conducted on a schedule such as up to every six or more months. The pre-scanning process can involve medical personnel that develop a strategy and plan to map an approach to advancing one or more needles into targeted vasculature through an acceptable path. The strategy or plan can include a small range of acceptable paths as well as a most desired path to vascular access. This plan can include mapping paths to numerous pre-determined cannulation sights which can then be accessed on a rotating basis via a rope ladder access technique. The acceptable cannulation sights and paths can all be determined after the pre-scanning process and prior to the patient undergoing dialysis. Accordingly, in one embodiment, the cannulation site and path to cannulation does not need to be calculated in real time-but can be all determined before the patient puts their arm in the detection bed.

In use, in one embodiment, the patient merely affixes their arm or other body part on the detection bed and the cannulation system then recognizes the patient and knows the acceptable path that the needle is to take to achieve cannulation. The cannulation system guides movement of a needle through the acceptable path thus preventing a needle from entering non-acceptable paths or insertion beyond a defined terminal point.

In one embodiment, the cannulation system is servo-controlled, wherein servomotors are attached to each moving part of the armature and sensors are associated with each servomotor such that the patient or other person can grasp a needle or needle holding fixture and move the needle through a path that is guided or constrained by the servomotors.

In another embodiment, the cannulation system is manually operated by a user and the user sets each movable part of the cannulation system according to the pre-determined plan. Here, the armature of the cannulation system lacks servomotors or sensors or other electronics.

In yet a further embodiment, once the body part is affixed to the detection bed and the patient is registered, the cannulation system operates autonomously to accomplish vascular access.

In one embodiment, a cannulation system includes structure and functionality to target vasculature and to position one or more needles within the target vasculature without the need for skilled personnel. The needle or needles are held within a needle retaining fixture and once the detection bed registers the patient, the armature is employed to advance the needle within the target vasculature or graft or fistula along a path calculated based on real-time fiducial or anatomical data and/or pre-recorded anatomical data. In one aspect, the needle or needles are advanced at a pre-determined angle and depth within a patient's body and within target vasculature. Significantly, the system is effective for providing vascular access and assists with cannulation throughout the body including specifically for radio-cephalic, brachio-cephalic and brachio-basilic fistulas and may range from the forearm to the upper arm or other locations on the body.

In one preferred embodiment, the system embodies a cannulation system that includes a detection bed equipped or associated with a body part affixing structure, and an armature including a needle retaining fixture attached or associated with the detection bed. Localization technology is embedded in the detection bed and is fixed in its relationship to the body part affixing structure and armature.

Once the patient's arm is stabilized onto the bed, the location of the fiducial(s) within the patient's arm or other body part is detected, which is linked via system software, comparing the fiducial(s) location to a previously recorded 3-D dataset which then can virtually map the location of the vascular access sites to the armature and where the needle tip is expected or directed to be positioned. The needle retention structure attaches to the needle in a fixed and reproducible way such that the tip of the needle is known (once secured into the needle retention structure) relative to all the anatomical landmarks via the system software. Once the needle is locked into the needle retention structure, with the data provided by the software, the proposed orientation of each of the degrees of freedom of the needle supporting structure is known. In a manual approach, these settings can be communicated by a display to the user and the user sets each joint or moving part of the armature. In a servomotor based approach, the various degrees of freedom of the armature is guided or limited by the servomotors to permit movement of the needle through the ideal path for each degree of freedom.

In a preferred embodiment, an arm of the patient is stabilized in a detection bed of a cannulation system, and the detection bed scans the fiducial(s) implanted in the arm to know an exact orientation of the arm within the cannulation system and relative to one or more component parts of the cannulation system. The position of the arm may be exactly as usual or expected, or the arm may be slightly rotated or offset from expected. The system adjusts the previously calculated and known path to take into account any slight differences in position of the arm whether the differences are translational or rotational or a combination thereof. The cannulation system makes such an adjustment either automatically or semi-automatically, or the adjustment is made manually with guidance. If there is a significant difference in position of the arm from expected, the system can alert the patient or healthcare provider. In this situation, the system can calculate a partially new or completely new path and trajectory based on the new information.

During the pre-scan, mapping and planning process, one or more of an MRI scan, a CT scan, ultrasound scan, infrared view, or 3-D photography is employed to collect information on a patient's vascular morphology and anatomy. In one embodiment, information concerning target anatomy and/or vasculature relative to implanted fiducial(s) is collected and stored within the cannulation system memory. In one particular aspect, the cannulation system embodies an array of passive limiters which fixes an extent of travel of each degree of freedom of the armature and in various approaches, information regarding the setting of each passive limiter is determined and displayed on a device using a combination of imaging data and information from real-time fiducials corresponding to the vascular access targets.

In various embodiments, the system can also track insertion sites, sizes, location and geometry, insertion dates, flow rates, treatment frequency and treatment length, as well as allows for patient input so that complications or infections are tracked and monitored. In certain alternative embodiments, the system can suggest insertion sites based upon a combination of historical data about the patient's previous cannula insertions with the device and the patient's anatomic imaging data, as well as in view of patient input, to provide options to the patient or healthcare provider concerning needle placement. In certain alternative embodiments, the system may also leverage crowd data from others of the system with similar anatomy and vascular morphology that had successful cannula insertion at a given site, in order to further inform the system's recommendation. Further, in alternative embodiments, the system includes a remote interface or computer that allows the patient, health care provider, or other connected health device (e.g. by Bluetooth) to enter patient health information including heart rate, blood pressure, and blood flow as well as patient diet, medication regiment, and exercise.

In alternative embodiments, the system controller manages or provides the assessment of a fistula or graft prior to cannulation. Should an obstruction be detected, the system will prevent a cannulation procedure and can alert the patient or health care provider so that further assessment and/or intervention can be conducted. Various sensors and actuation mechanisms are provided to automate the assessment process, or portions thereof. In certain approaches, the system embodies one or more sensors that recognize thrill or vibrations, that operate like a stethoscope to track for bruit (i.e. sounds of heartbeats or blood flow), or listens to flow to look for obstructions.

In one or more aspects, an access system includes one or more fiducials or markers that facilitate introduction of a needle into a patient's skin relative to a vascular access site. In alternative embodiments, the fiducial or marker can be one or more implants, tattoos, magnetic marker, radio-opaque materials, or other features naturally occurring in areas near or at an intended vasculature access site.

In a preferred approach, fiducial markers are configured to be implanted and attached to the radius and/or ulna bones of the forearm or placed in one or more locations into the bony or soft tissue near the vascular access sites. The vasculature access site fiducial or marker can be co-registered to the previously acquired infrared view, MRI, ultrasound, or CT scans to initially set up patient data for subsequent cannulation.

In alternative embodiments, the system can be configured to readjust trajectory and/or depth of needle movement should the patient move before or during or between cannulations. Moreover, the fiducial or marker can alternatively or additionally be one or more RFID chips, electro-magnetic, metallic implants, radio-opaque material, or other features naturally occurring in areas near or at an intended vasculature access site.

In another alternative approach to inserting a needle within a vascular access site, there is provided a sensor pack that can both locate as well as assess a fistula or graft prior to cannulation. The sensor pack can be attached to an automated mechanism that moves along the arm or can be embodied in a sleeve or other structure that the patient can move along the insertion site. Information gleaned by the sensor pack is fed into the system to facilitate determining the desired cannulation location. Additionally, the sensor pack can be configured to continue to assess the status of the cannulation and communicate the same to the patient or healthcare professional.

In one or more alternative approaches, the patient can move the components of the cannulation system and advance the needles themselves, taking into account their own sensation, pain, or comfort, where the system ensures that the needle does not take an incorrect path or be inserted to deeply beyond a defined and desired terminal point. Additionally, in each or one or more alternative approaches of the disclosed embodiments, the system can automatically position, aim and advance a needle in a cannulation procedure. Further, in alternative embodiments and approaches, the patient and the system work together to varying degrees and in various ways such as where the patient lets the system guide positioning the needles but wants to push the needles into vasculature, or where the patient moves the structure holding the needles and lets the system push the needle into vasculature either when the system is ready or when the patient indicates they are ready and signals that cannulation should be performed such as by engaging a cannulate button.

In one embodiment, the cannulation system includes a sleeve or chamber positionable over a patient's arm or in which the patient's arm is inserted. The sleeve is configured to lock onto the patient's arm and facilitates fixing the patient's arm relative to the detection bed. In another approach, the sleeve or chamber includes a molded insert contoured to accept and position the patient's arm relative to the detection bed, the molded insert being created via 3-D printing from a model of the patient's arm, for example, so the contours of the mold allow the arm to sit firmly and comfortably in the molded insert. The system can communicate to the detection bed to adjust the position of their arm.

In various additional alternative aspects, the cannulation system can include one or more non-visible light, acoustic, pressure or visual sensors for determining and tracking blood flow. In this way, the effectiveness of a cannulation as well as the health of the patient can be confirmed and monitored. Also, the system can alternatively or additionally employ acoustics, for example ultrasound, or audible triangulation, or wavelengths of light, for example near-infrared imaging, to locate or identify and target vasculature. For example, an IR sensor can be so positioned and configured to emit near IR beam that identifies the targeted fistula or cannulation site. Thus, in various approaches, one or more points of access within an AV fistula or graft can be suggested or selected by the alternative approach to the system, healthcare provider or patient. Moreover, in the alternative approach, the system can indicate whether a cannulation is successful and alert the patient or healthcare professional regarding the success or failure of a cannulation. Also moreover, in another embodiment, the system can alternatively or additionally employ acoustics, for example ultrasound, or audible triangulation, or wavelengths of light, for example near-infrared imaging to identify the patient. Furthermore, acoustics or wavelengths of light of can be used to register the system in relation to the target vasculature.

Further, in other aspects, various approaches to cannulation systems are provided with structure and functionality to controllably position one or more needles in three-dimensional space and relative to a target insertion site. In one approach, a cannulation system includes one or more standard needle cassette assemblies that can be configured to be controllably translated along an armature including a rotatable U-frame that is arranged to position the needles in three-dimensional space and relative to target insertion sites. In an alternative embodiment, an armature including an articulating split frame is provided to independently position two or more needles relative to an insertion site.

Various other alternative approaches to armature structures can additionally be used so long as effective cannulation is achieved. For example, a curved arm that supports a needle cassette and that traverses in an arc path or structures that include ball joints or other approaches to provide multi-axis movement can be employed.

These and other features of the disclosure will become apparent to those persons skilled in the art upon reading the details of the systems and methods as more fully described below.

### BRIEF DESCRIPTION OF THE DRAWINGS

FIG. 1 is a schematic representation, depicting a hemodialysis procedure and equipment.

FIG. 2 is a top view, depicting a graft in a first vascular access approach.

FIG. 3 is a top view, depicting an AV fistula in a second vascular access approach.

FIG. 4 is a top view, depicting cannulation of the second vascular access approach.

FIG. 5 is a top view, depicting the flow of blood resulting from cannulation.

FIG. 6 is a cross-sectional view, depicting one approach to a fiducial.

FIG. 7 is a partial perspective view, depicting a device for implanting fiducials within tissue.

FIG. 8 is a schematic representation, depicting anatomy containing implanted fiducials.

FIGS. 9 and 10 are side views, depicting the motion of bones of a forearm.

FIG. 11 is a side view, depicting attaching a marker to a bone.

FIG. 12 is a schematic view, depicting a scanning system.

FIG. 13 is a side view, depicting a vessel and a controlled needle insertion path.

FIG. 14 is a perspective view, depicting one approach to a cannulation system.

FIG. 15 is a perspective view, depicting another approach to a cannulation system.

FIG. 16A is a perspective view, depicting yet another approach to a cannulation system.

FIG. 16B is a perspective view, depicting the system of FIG. 16A being used on a patient's arm in combination with a stabilizer device.

FIGS. 17-19 are perspective views, depicting use of a cannulation system.

FIGS. 20-21 are perspective views, depicting another approach to a cannulation system.

### DETAILED DESCRIPTION OF THE DISCLOSURE

Before the present systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described.

It must be noted that as used herein and in the appended claims, the singular forms “a”, “an”, and “the” include plural referents unless the context clearly dictates otherwise. Thus, for example, reference to “the system” includes reference to one or more systems and equivalents thereof known to those skilled in the art, and so forth.

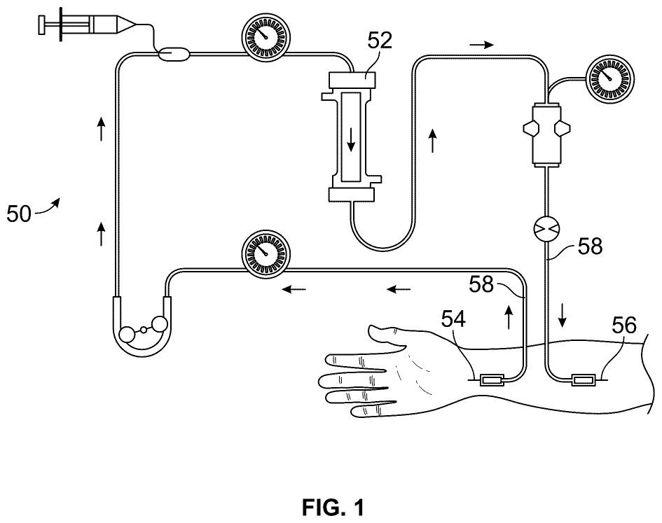

With reference to FIG. 1, a hemodialysis system 50 and associated equipment is shown. During hemodialysis, the patient's blood is routed through a dialyzer 52 which filters the blood. The patient is prepared by cleaning the sites from which blood flow out and then back into the patient's blood vessels. As described more below, steps are taken to prepare the patient's vasculature for dialysis. Once the patient's vessels are so prepared, at the start of hemodialysis, a pair of needles 54, 56 are inserted into the patient's arm. A numbing compound can be used to facilitate needle insertion and to minimize pain. Each needle is attached to a soft tube or cannula 58 that connects to the dialysis system 50. The dialysis system 50 pumps blood through a dialyzer 52 and returns the blood to the patient's body. During this process, blood pressure is monitored and controlled by the dialysis machine thereby controlling the flow of blood through the filter and speed of which blood flows from and to the patient.

As blood enters the filter, it is forced through a large number of thin, hollow fibers. At the same time, a dialysis solution passes in the opposite direction about the fibers. Waste products are thereby removed from blood and carried by the dialysis solution. Filtered blood is then returned to the patient's vasculature. In this way, extra salt, potassium, calcium and fluid is removed from blood.

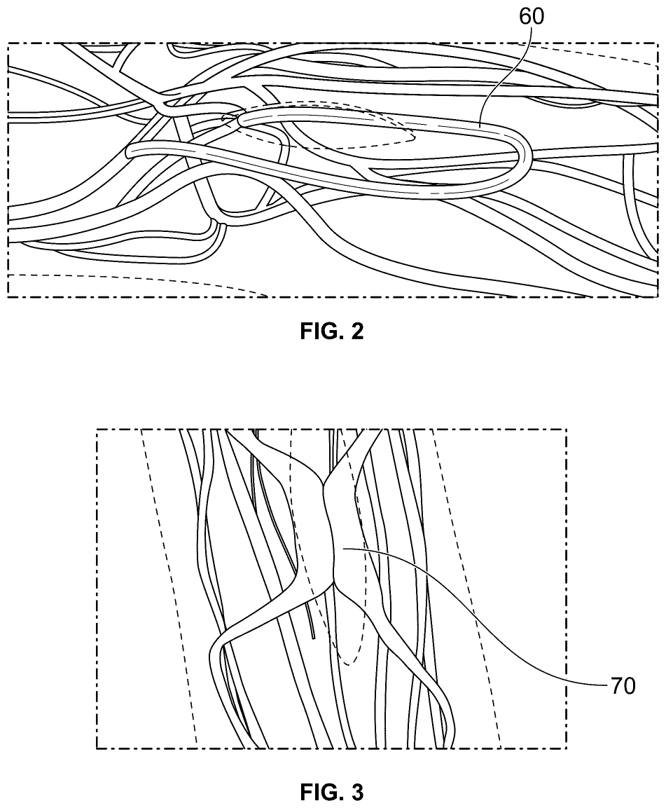

An important step before starting hemodialysis treatment is having surgery to create a vascular access site. Vascular access is a phrase used to describe the place on a patient's body where blood flows from and returns to the patient's vasculature such as during hemodialysis. A hemodialysis vascular access site may be a catheter, an arteriovenous (AV) graft 60 (FIG. 2) or an arteriovenous (AV) fistula 70 (FIG. 3). Notably, the catheter approach is generally employed for temporary access and not suited as a permanent solution for home or treatment center dialysis.

To create an AV graft 60 (FIG. 2), during an outpatient procedure, a surgeon cuts the skin to gain access to target vessels. The surgeon then uses a synthetic tube graft 60 to connect an artery which carries blood away from the heart to a vein which carries blood to the heart. The surgical site is then closed, leaving the graft available for dialysis. Dialysis needles are then repeatedly used to access the tube during a hemodialysis procedure as described above. Generally, the AV graft approach is employed for patients that have veins that prevent them from having an AV fistula since the AV graft approach is more often associated with infection and repeated blood clots that can block the flow of blood and make it hard or impossible to have dialysis.

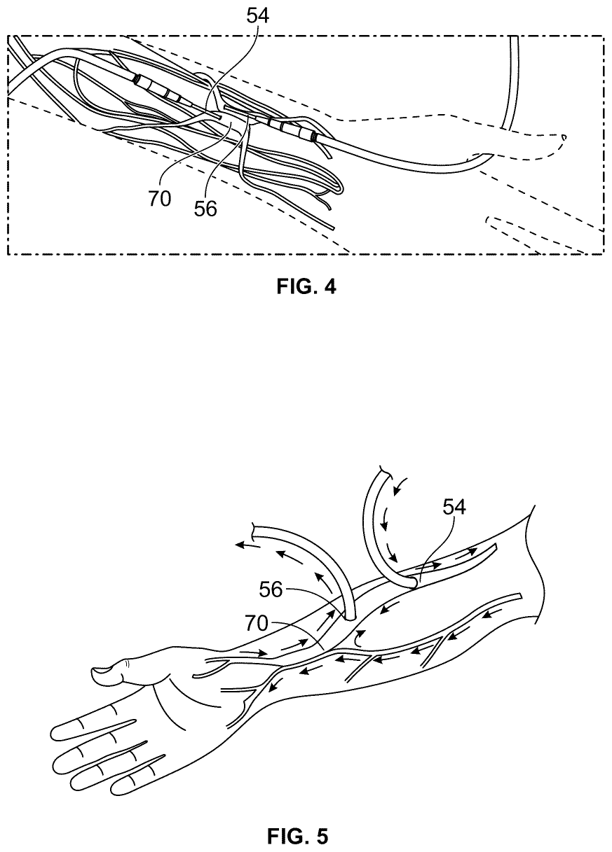

The generally accepted best type of long-term vascular access is an AV fistula 70 (FIG. 3). This approach is characterized as providing the highest blood flow for dialysis, is less likely to become infected or clot, and as lasting longer than other approaches to vascular access. Here, the surgeon connects an artery directly to a vein, usually within the patient's arm, to create the AV fistula. When the vein is so connected to the artery, the vein grows wider and thicker, making it easier to repeatedly place the needles for dialysis. The AV fistula itself also has a large diameter that allows blood to quickly flow out and back into the patient's body, the goal being creating a system where there is high blood flow so that the largest amount of blood can pass through the dialyzer. FIG. 4 shows the cannulation of two needles 54, 56 at an AV fistula site 70, and FIG. 5 depicts the flow of blood to and from the AV fistula 70 during dialysis. It is to be noted that in using the presently disclosed cannulation systems, any number of treatment protocols can be specified by the treating healthcare professional and embedded into software such that needle cannulation sites may be rotated in a rope ladder technique or repeatedly used as in the buttonhole technique.

Once the patient is provided with a vascular access site, the challenge becomes placing the needles within the site. As stated, many patients find it difficult or impossible to self-cannulate. In addition, inexperienced nurses find it difficult to cannulate.

Accordingly, various approaches to access methods and apparatus are presented. The disclosed approaches are configured to provide a repeatable, effective and exacting approach to self-cannulation or healthcare provider assisted-cannulation. The disclosed approaches are intended for use in a center setting or a home setting for hemodialysis.

The disclosed cannulation systems are configured and function to position a needle or needles and, in some embodiments, advance the needle within the targeted AV graft, fistula, vessel or vessels. The system further includes functionality for positioning a needle into the correct position and trajectory and, in some embodiments, to advance the needle at a pre-determined angle and depth within a patient's body and within target vasculature. The system is effective for providing vascular access and assists with cannulation throughout the body including specifically for radio-cephalic, brachio-cephalic and brachio-basilic fistulas and use may range from the forearm to the upper arm or other locations on the body. The system can be easily operated by the patient, unskilled personnel or skilled healthcare workers in a home setting or in a treatment center and functions to cannulate the patient successfully while improving outcomes and reducing complications.

As stated, as part of a pre-scanning process, one or more of an MRI scan, a CT scan, an ultrasound scan, infrared view, or 3-D photograph can be employed to collect information on a patient's specific anatomy and vascular morphology. In one embodiment, information concerning target cannulation sites can be collected or stored within cannulation system memory. This input data on patient's anatomy and vasculature is acquired (e.g. the anatomic MRI scan, ultrasound scan, CT scan, infrared view, or 3-D photography) to register a patient and to map an approach to cannulation. The cannulation system controller including system software and/or firmware uses such stored patient data to then manage the operation of the structure guiding cannulation needles.

In one or more alternative embodiments, the system controller tracks one or more of insertion sites, sizes, location and geometry, insertion dates, flow rates, treatment frequency and treatment length, and is responsive to patient health data so that complications or infections are tracked and monitored. The system can then suggest insertion sites based upon patient usage data, anatomic characteristics, and vascular morphology, and provides options to the nurse or patient concerning needle placement. In an alternative embodiment, remote interface or computer is included and is configured to allow the patient or health care provider to enter patient health information including heart rate, blood pressure, and blood flow as well as patient diet, medication regiment, and exercise. This patient health information may also be collected passively through connection with patient's wellness devices (e.g. via Bluetooth) or integration with electronic health record systems.

In one alternative embodiment, the system provides for the assessment of a fistula or graft prior to cannulation. In this regard, such functionality can be fully or semi-automated and communication of or an alert concerning the assessment conducted by the system to the patient and/or the healthcare professional can be further provided. Should an obstruction be detected, the system will prevent a cannulation procedure and can alert the patient or health care provider so that further assessment and/or intervention can be conducted. Various sensors and actuation mechanisms are provided to automate the assessment process, or portions thereof. The sensors can be one or more of acoustic, tactile, ultrasound, doppler, infrared, near-infrared or the like.

In certain alternative approaches, the system embodies one or more sensors that recognize thrill or vibrations associated with the cannulation target site, that operate like a stethoscope to track for bruit (i.e. sounds of heartbeats or blood flow), or listens to flow to look for obstructions. In one or more approaches, sensors are provided to feel for thrill or listen for bruit, the sensors being configured to listen for normal, unobstructed flow or for indications of stenosis or clots. The sensors can also be equipped to look for other indications of normal flow or obstruction such as issues associated with the Steal Syndrome and alert as necessary. In one embodiment, a bank of sensors can be provided in a package that is configured to be translated either manually or automatically along a cannulation target site. This package can be embodied in a separate cuff or a structure resembling a stethoscope head, or can be attached to or comprise a portion of an arm or other structure of a needle support assembly. The system itself or the patient can be prompted to place the sensor package at the cannulation target site and to direct movement of the sensor package to assess the target site. In one approach, positioning of the sensor package can be dictated or controlled by using the below described fiducial markers.

Thus, in certain alternative embodiments, the location of the graft or fistula can be ascertained and confirmed by employing the sensor package. Whether it is moved automatically or manually, real time assessment can confirm the location and condition of the graft or fistula. The system can thus be configured to readjust trajectory and/or depth of needle movement should the patient move before or during or between cannulations. The sensor package can be tracked by the same system that tracks the needle cassette and the system observes and tracks the sensor locations relative to when the vessel is properly detected. This information can be employed to confirm the location and condition of the cannulation site and also be fed into the system's database to help determine and plan the location of the cannulation site along the graft or fistula.

In one or more additional or alternative embodiments, the access systems can include one or more non-visible light, acoustic, pressure or visual sensors (not shown) for determining and tracking blood flow. The location of maximum flow can be identified by the pressure sensor so that the needle is placed in the optimal location. The disclosed systems can also alternatively or additionally incorporate acoustics (e.g. ultrasound or audible triangulation) or a light source providing wavelengths of light to locate or identify and target vasculature (e.g. near-infrared). One or more points of access within an AV fistula or graft can be suggested or selected by the system or patient. In these ways, the effectiveness of a cannulation as well as the health of the patient can be monitored over time.

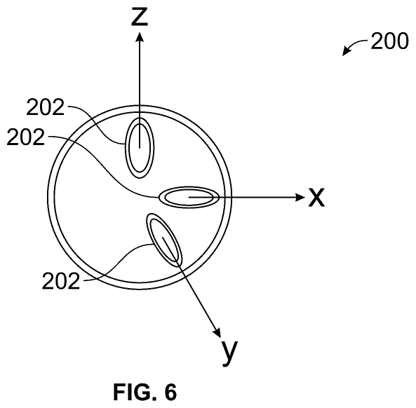

With reference to FIGS. 6-8, there is shown an approach to fiducial markers for use in registering a patient and for use in the pre-scanning process to develop a cannulation plan or map. It is to be understood that fiducials may be magnetically or electrically detectable but also imageable using electromagnetism, MRI, CT or ultrasound so that their location relative to the target vessel may be determinable.

As shown in FIG. 6, in one approach, a fiducial can be a bead 200 that includes one or more coils 202 that are configured within an interior of the bead 200. In one particular aspect, three coils 202 are configured and arranged so that they provide information concerning the orientation of the bead 200 in three-dimensional space such as relative to x-y-z orientational axes, as well as concerning pitch, roll and yaw. That is, each coil provides positional information regarding one axis, and in some embodiments, pitch, roll and yaw. Ideally, fiducials are insertable without the need for surgery, but they may be surgically placed as well when the vascular access site (e.g. AV graft or fistula) is made.

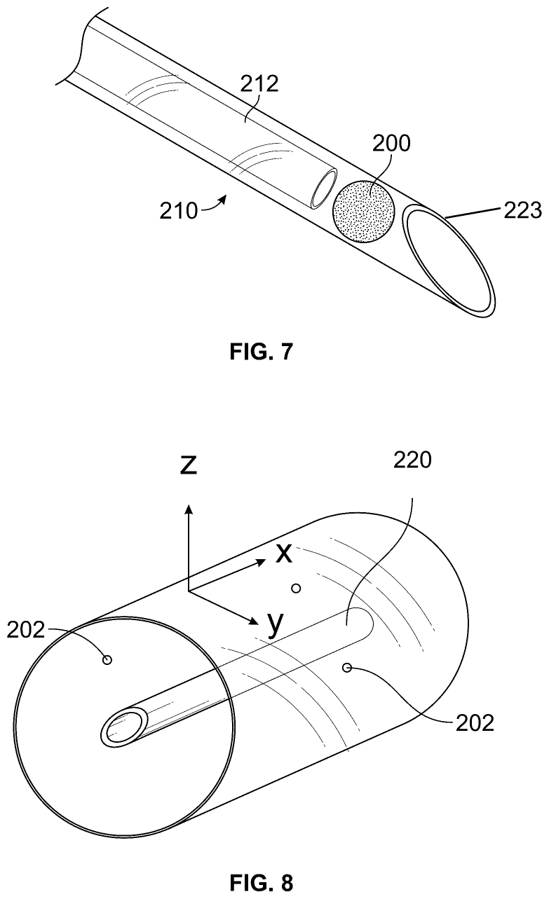

In one approach (FIG. 7), an elongate assembly 210 can be employed to insert a fiducial bead 200. A plunger 212 is configured within the needle assembly 210 and arranged to be longitudinally translatable within the elongate assembly 210 to advance one or more beads 200 out of a terminal end of the elongate assembly 210. In an apparatus configured to insert multiple fiducials, a terminal end of the apparatus can be configured to deliver a single fiducial at a time, such as by including a chamber or equivalent structure for registering a single fiducial for delivery while including additional structure retaining additional fiducials for subsequent delivery. Three fiducials may be required to be implanted near an insertion site relative to a target vessel 220 (see FIG. 8), or if the fiducials 200 themselves embody known three dimensional shapes or orientations, only one or two fiducials may be required to assist in locating an insertion site on a particular patient. Moreover, when using an electric field in combination with fiducials, each fiducial may contain one to three coils tuned to different resonant frequencies such that their orientation and location can be determined. Thus, once scanned in-situ using electromagnetism, MRI, CT or ultrasound, a data set is created to precisely locate anatomical structures in three dimensional spaces based upon the relative location of the fiducials.

After the fistula or graft is made and stabilized, or targeted vasculature is identified, the fiducial(s) may be placed in a follow-on procedure on or adjacent the fistula or graft, or as described below, a bony position. During this procedure, the healthcare provider defines optimal trajectory for the cannulation and needle path based upon the relative location of the fiducial(s) to the targeted cannulation site. The cannulation system functions to closely replicate this healthcare provider-set, pre-determined position and trajectory path relative to the implanted fiducial at the vascular access site. The fiducials are detected by the access system so that the position of target vessels or grafts is located relative to the fiducials. Pitch, roll and yaw and x-y-z axis positioning of a fixture supporting a standard needle set is managed by the cannulation system so that the needle set is moved within an acceptable access path. The cannulation system thus conveniently operates to constrain or manage movement of a needle to the acceptable path to prevent the needle from entering non-acceptable paths or insertion beyond a defined terminal point.

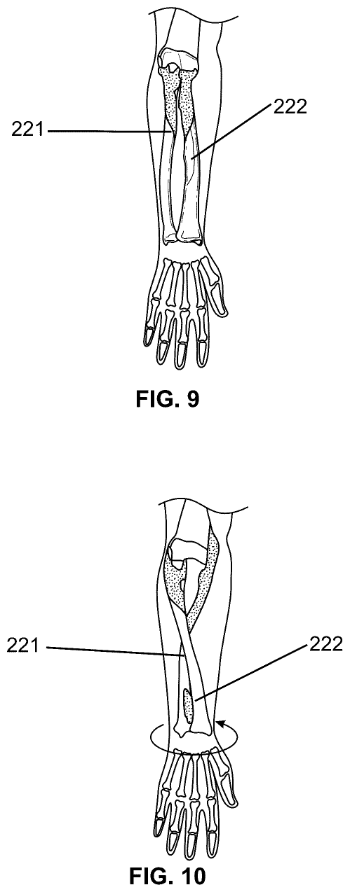

In one particular approach, fiducials are implanted and attached to one or more of the ulna and radius bones of the forearm. As shown in FIGS. 9 and 10, the ulna 221 and radius 222 bones of the forearm move with respect to each other when the forearm is rotated. However, the ulna 221 and radius 222 bones are nevertheless a useful location for attaching the fiducials 200 and provide a reliable reference with respect to the vasculature targeted for dialysis needle insertion when the forearm is held in a position expected or dictated by the access system.

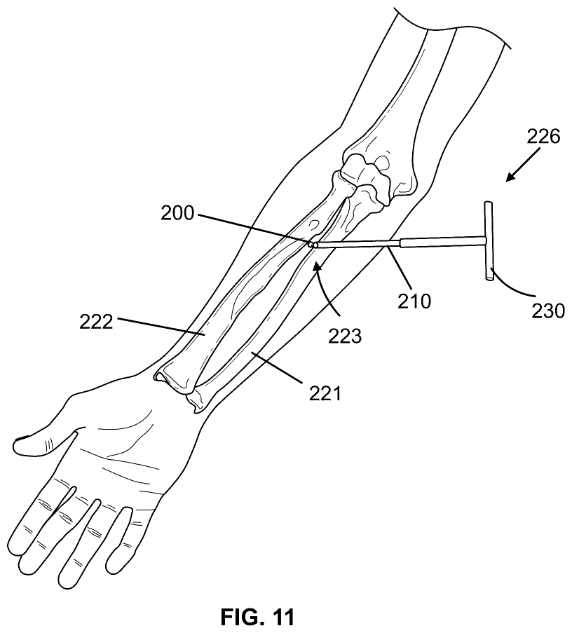

With reference to FIG. 11, an insertion device 226 is employed to pierce the tissue of the forearm and to attach one or more fiducials 200 to one or both of the ulna 221 and radius 222 bones. The insertion device 226 includes an elongate shaft 210 extending from a handle 230. A terminal end 223 of the shaft 210 defines a needle point and houses the plunger 212 (FIG. 7) that is configured to advance one or more fiducials 200 out of the terminal end 223 and into engagement with the ulna 221 and radius 222 bones (See also FIG. 7).

As stated above, depending on the type of fiducial being employed and the location that the fiducial is implanted, one to three fiducials can be implanted. The handle 230 is configured to be used to advance the device 226 through tissue and to create a cavity in bone or otherwise prepare the bone for receiving a fiducial. The handle 230 is connected to the plunger 212 to cause longitudinal motion of the plunger 212. For example, the handle 230 can be locked during advancement of the insertion device 226 through tissue and to engage bone and create an implantation site for the fiducial 200. The handle 230 is then unlocked, such as by rotating the handle a quarter turn, so that the handle 230 can be moved longitudinally to advance the plunger and consequently move the fiducial 200 into fixed engagement with bone.

Once fiducials are placed as desired, the forearm or other body part including implanted fiducial(s) is scanned using a method configured and arranged to create a three-dimensional image. This will link the fiducial locations to target vessel locations, relative dimensions, and trajectory. Using this information, cannulation can then be performed to facilitate accessing target vessels.

In an alternative embodiment, the cannulation system includes structure and functionality to determine if one or more implanted fiducials have migrated. The system detects when a fiducial is not where it is expected to be and assesses whether the displacement of the fiducial is significant and/or will impact the pre-determined path to cannulation. For example, the system can measure the displacement of the fiducial in three-dimensions and record the displacement information. The displacement information is then analyzed to determine whether a new or adjusted canulation path is necessary. The cannulation system can also alert the user or healthcare provider so that they are aware of the migration and can consider whether a new or adjusted path is necessary as well as to consider whether other steps should be taken such as removing and/or replacing the migrated fiducial(s).

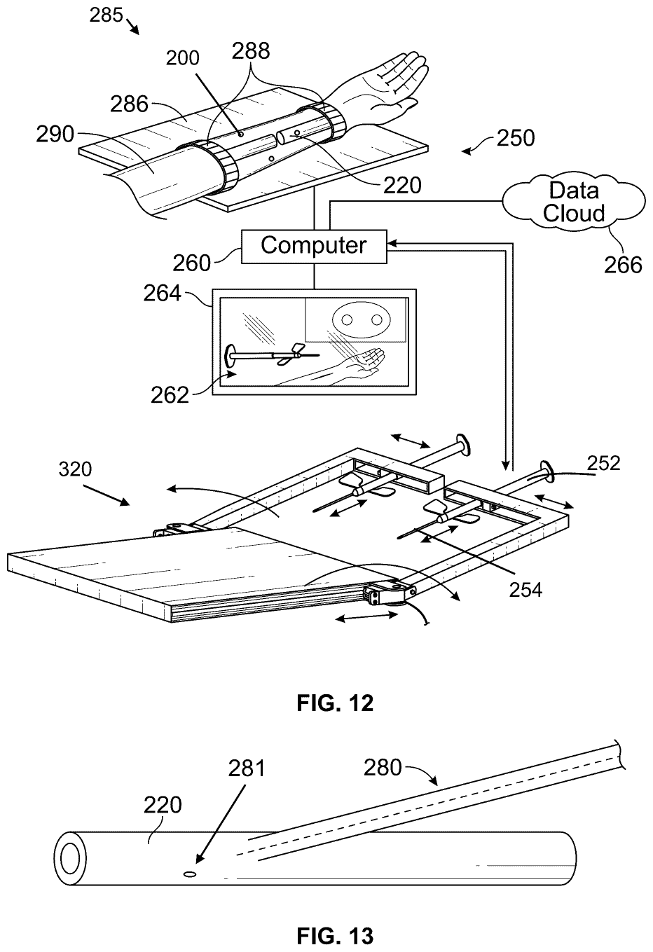

In one approach, a scanning system 250 (FIG. 12) is employed to plan the placement of a vascular access set or kit 252 including a needle or cannula 254 within a target vessel 220 such that it may be entered safely at a correct angle and depth. Accordingly, the scanning system 250 is configured to create an image, for example a three-dimensional image 262 on a user accessible computer interface 264 by employing an MRI scan, a CT scan, ultrasound scan, infrared view, 3-D photography or other scan to collect information on a patient. Here also, information concerning target anatomy and/or vasculature is collected or stored within system memory, such as within a data cloud 266.

Based upon the pre-scanning process, the cannulation system 320 is provided with a map or plan for positioning the vascular access set 252. In certain embodiments, the scanning system 250 is employed to suggest insertion sites based upon patient history, system usage data (e.g. previous successful insertion trajectories), and input, and provides options to the patient concerning needle placement into the target vessel insertion site 220. In one aspect, the system can manage, but is not limited to, rope ladder or buttonhole site strategies for cannulation as necessary or dictated by the system or health care professional.

Further, in an alternative embodiment, the scanning system 250 can be configured to allow the patient or health care provider to enter patient health information including heart rate, blood pressure, and blood flow as well as patient diet, medication regiment, and exercise, or other data that can have a bearing on effective cannulation. The system may include internet and Bluetooth connectivity. This data may also be entered passively through connected wellness devices or electronic health record systems.

In one particular approach, the scanning system 250 includes a scanning assembly 285 including a bed 286 and further includes securing means 288 for retaining, in a repeatable and precise manner, an arm or other body part 290 relative to the bed 286. Various approaches to securing means are contemplated with the objective of maintaining a precise orientation of the body part relative to the bed 286. That is, various attachment devices, sleeves or molded body part receivers can be employed as securing means. Moreover, the bed 286 can be a simple platform or can be a detection bed that is embodied in a cannulation system as described below. In either case, the bed used in a scanning procedure holds or retains the scanned body part in precisely the same orientation as is accomplished by a detection bed of a cannulation system.

Once the body part (such as an arm) 290 is placed on the bed 286 and secured, an MRI scan, a CT scan, ultrasound scan, infrared view, 3-D photography or other scan is conducted to collect information used to define and map an acceptable access path for cannulation that prevents the needle set 252 from entering non-acceptable paths or insertion beyond a terminal point 281 (FIG. 13). This acceptable path may be defined by the physician or mathematically modeled based on the patient's anatomy and the implanted fiducials. The acceptable path may also be progressively defined across treatment sessions using machine learning algorithm applied to the trajectory data of previous successful insertions for the given patient, and can include the angle for entering a targeted vessel or graft as well as a path that avoids certain other anatomy.

The scanning system 250 is thus configured and functions to plan or map placing a needle or needles within to the access site, AV graft or fistula. The goal here is to provide a patient with a self-cannulation or healthcare provider assisted-cannulation system that at least defines an acceptable access path 280 to and within a target vasculature 220 (See FIG. 13) and prevents a cannulation operator from entering non-acceptable paths or insertion beyond a defined terminal point 281.

In an alternative embodiment, a management system can be further provided and can also be in communication with dialysis equipment to further provide feedback concerning the success of the cannulation. In alternative embodiments, automated or medical personnel generated messages concerning the same can be provided to the patient as well as suggestions regarding any necessary interventions such as a need to re-cannulate. Telehealth video interface for real-time feedback through internet connection may be included. Trajectory limits may also be defined based on anatomic structures defined by the patient's ultrasound scan, infrared view, MRI or CT or other imaging, so that trajectories that place the needle in sensitive or non-target anatomy are not allowed by the system.

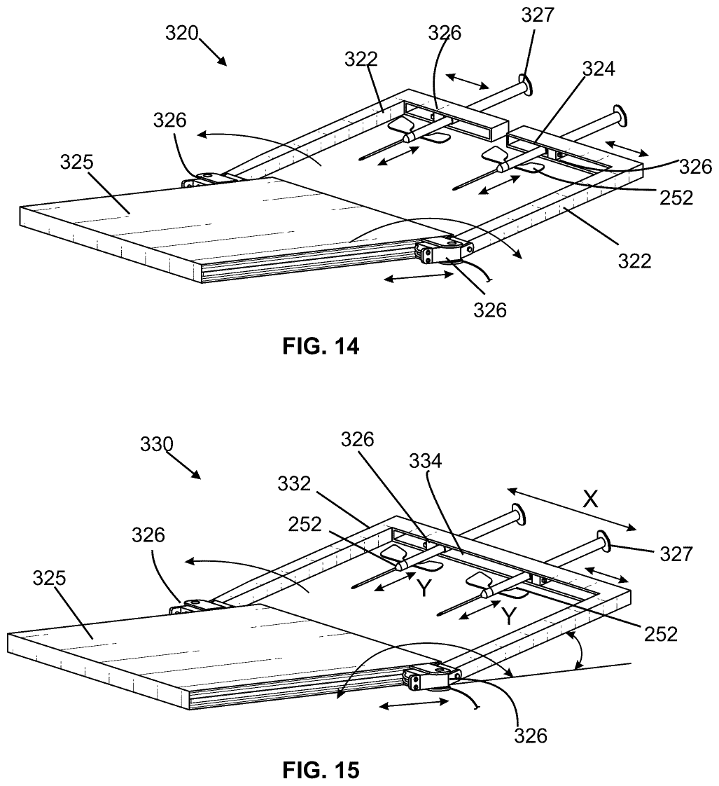

With reference now to FIG. 14, one approach to a cannulation system 320 can embody an armature including a pair of articulating L-shaped frames 322 that each include a track 324 along which a standard access kit 252 can be attached and configured to be translated. It is to be recognized that the cannulation system 320 need not include such articulating frames but can embody one or more of the features of the other approaches disclosed herein or the equivalent thereof, so long as an access kit or other needle assembly can be controllably manipulated to effectively cannulate a graft or fistula. Moreover, the cannulation system can include joints or other movable parts that each only have a single degree of freedom to thereby greatly simplify the math associated with software or autonomous control of an armature to choose the best path towards an objective, as well as simplify user interface complexity. Moreover, needles of various manufacturers or sources can be recognized by the system such as reading a bar code or other identifying information and the cannulation procedure can be adapted for different needle sets.

In use, the patient simply affixes their arm or other body part within the cannulation system which recognizes the patient and knows the acceptable path that the needle is to take to achieve cannulation. The cannulation system guides movement of a needle through the acceptable path thus preventing a needle from entering non-acceptable paths or insertion beyond a defined terminal point.

In one preferred approach, the cannulation system is servo-controlled, wherein servomotors are attached to each moving part of the armature and sensors are associated with each servomotor such that the patient or healthcare personnel can grasp a needle and move the needle through a path that is guided or constrained by the servomotors. Here, the sensors detect precise angular or translational locations of components of the armature. In another preferred approach, the cannulation system is manually operated by a user and the user sets each movable part of the cannulation system according to the pre-determined plan. Here, the armature of the cannulation system lacks servomotors or sensors or other electronics. In yet a further approach, once the body part is affixed to the detection bed and the patient is registered, the cannulation system operates autonomously to accomplish vascular access.

In a preferred embodiment, a controller of the cannulation system embodies an array of passive limiters which fixes an extent of travel of each degree of freedom of needle supporting structure and in various approaches, information regarding the setting of each passive limiter is determined and displayed on a device using a combination of imaging data and information from real-time fiducials corresponding to the vascular access targets. It is noted that scans can be performed periodically so that the positional relationship of fiducials to target grafts or vessels is confirmed or modified based on changing conditions including selectively changing access insertion areas. Also, an operator may move the needle into place with the system simply limiting excursion from a most desired needle path.

In an alternative approach, the system may provide haptic feedback so that if the human operator begins to drift from the optimally defined trajectory, they feel resistance and can correct back to the intended trajectory within acceptable tolerance.

Although it is contemplated that standard needle set be employed, in various alternative embodiments, needle options can be provided to a user based upon patient anatomy or preference. In this context, the system can be equipped with functionality to know the difference between needles so that the location of a needle tip can be determined. Information on the needle of choice can be manually entered into the system or scanned via barcode or the like during workflow. A multitude of means for inserting a needle into the cannulation system can also be provided such as actuating a button or joystick or other input system that will advance the needle via patient or other user control. The system may also allow an ability to advance the needle incrementally (once touching the skin for example) or allow options for speed of entry. Such functionality can be based upon patient preference, all within safety guidelines of the system including knowing maximum distance of entry and maintaining the desired pathway. Additionally, the user may be able to physically advance the needle but is constrained by the system to ensure the needle does not go too far or off track. Here also, the system can provide haptic feedback, sufficient stability and dampening as the user advances the needle.

In the embodiment shown in FIG. 14, a body part holding or affixing apparatus is attached to or incorporated into a detection bed 325 of the cannulation system 320 and the articulating L-shaped frames 322 are attached to the bed 325. Localization technology is embedded in the bed 325 of the cannulation system 320 and is fixed in its relationship to the holding apparatus.

Once the patient's arm is stabilized onto the bed 325, the location of the fiducial(s) within the arm is detected, which is linked via system software, comparing the fiducial(s) location to a previously recorded 3D dataset which then can virtually map the location of the vascular access sites to the armature and where the needle tip is expected or directed to be positioned. A needle retention fixture attaches to the needle in a fixed and reproducible way such that the tip of the needle is known (once secured into the needle supporting structure) relative to all the anatomical landmarks via the system software. Once the needle is locked into the needle supporting structure, with the data from the software, the proposed orientation of each of the degrees of freedom of the needle support structure is calculated.

In another preferred embodiment, these settings can be communicated by a display to the user and set at each joint or moving part of the needle support structure by the user manually, or the degrees of freedom can be guided by servomotors to guide the user into the desired path for each degree of freedom without any manual entry.

Accordingly, real-time positional relationships of the fiducials relative to access kits 252 can be observed and compensated for by employing an electromagnetic energy-based navigation system incorporated into the detection bed 325 which identifies and tracks the location of the fiducials such as those implanted in bones of the forearm. A pre-determined positional relationship between fiducials and cannulation system armature structure is ensured by securing the arm or other limb to the bed 325. Such a platform can be the same or equivalent apparatus as that used when obtaining an (CT or MRI or other) image of target vessels with respect to implanted fiducials and that is inputted into the access system 250. The securing means as described above is employed to ensure an expected positional relationship, required by the access system 250 or as developed from CT or MRI scanning for example, between the body part implanted with fiducials 200 relative to the moveable access kits 252.

In one preferred approach, servomotors 326 are configured to permit movement of the access kits 252 in two directions—along a base of the L-shaped frames 322 and perpendicularly to the base of the L-shaped frames 322. Sensors are associated with each servomotor so that the positioning of the various moving parts of the armature can be known.

In another preferred approach, the cannulation system lacks any electronics and positioning of the needle is accomplished manually. Manual operation of armature of such a cannulation system can be guided by the pre-scanning plan, which instructs the user to place the moving parts of the cannulation apparatus at certain angles or positions. Additionally, the system would provide the user with instructions regarding the manner and sequence in which the various components supporting the needle should be moved and placed to ultimately accomplish the targeted cannulation. Here, angular or longitudinal markings and means for locking the moving parts can be provided on the cannulation apparatus to aid in proper positioning of the access kits relative to targets.

Each access kit 252 can be equipped with a stopper 327 to limit the movement of the access kit 252 in a direction perpendicular to the base of the L-shaped frame 322 generally or as determined by the terminal insertion depth point of a vessel. As stated, the movement of the component parts of the armature can be performed manually as prescribed by the system. Also, as stated, servomotors 326 can also be provided at the connections between the L-shaped frames 322 and the bed 325 to provide both control of rotational motion of the L-shaped frames 322 relative to the bed 325 as well as to control translation of the L-shaped frames 322 along the bed 325. In this way, the access kits 252 can be positioned and advanced as dictated by the pre-scanning plan.

In certain approaches, one or more of the servomotors can be omitted or configured to be selectively operated so that certain of the steps of moving or positioning the access kits 252 can be conducted manually. For example, the motors would be configured to support the weight of the system allowing the user to easily manipulate the needle support structure in a smooth manner, but also be configured to resist movement through an undesired path. Here, haptics can be employed and adjusted based upon the speed or mode of fixture movement by the user.

As shown in FIG. 15, the armature of the cannulation system 330 can alternatively be embodied in a single U-shaped articulating frame 332 having tracks 334 along which access kits 252 can be configured to be translated. Servomotors 326 can be provided to control the motion of the U-shaped frame 332 relative to the bed 325 and the access kits 252 relative to the U-shaped frame 332. Not shown are means for securing a body part implanted with fiducials to the bed 325. Here too, the system 330 can lack electronics and action of the various components of the armature can be manually controlled to thereby position the access kit relative to the cannulation site.

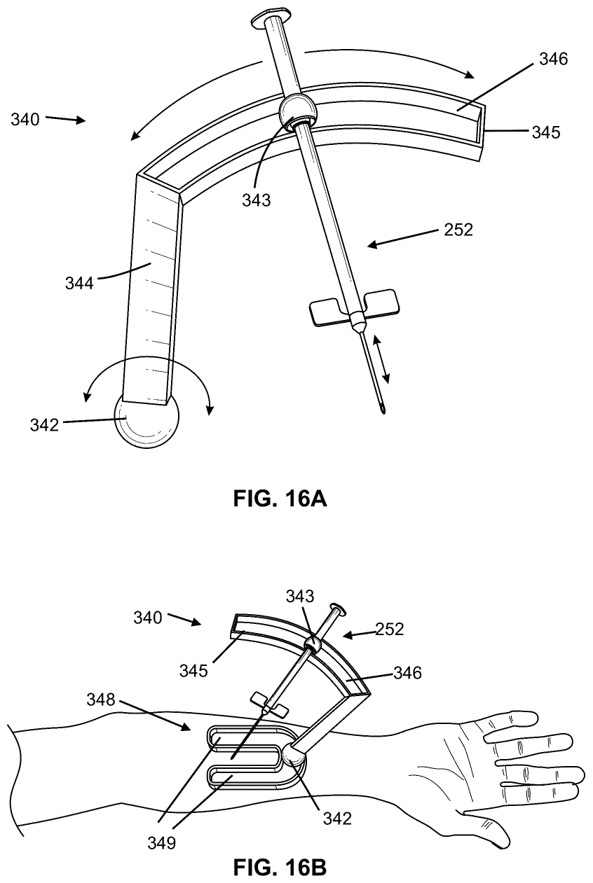

With reference to FIGS. 16A-B, another alternative approach to a cannulation system 340 includes structure that manipulates and positions an access kit 252 relative to a targeted insertion site. Although a single access kit 252 is shown attached to the cannulation system 340, the apparatus can be configured to support two access kits 252. Moreover, as with the other contemplated cannulation systems, movement of the various components can be servo-controlled or manual, or a combination thereof.

In one approach, ball joints 342, 343 are configured at articulation points to provide the apparatus with an ability to move the access kit 252 as dictated by the cannulation system or alternatively, manually by a user such as a patient or health care provider. Servomotors can be configurable at the ball joints 342,343 to control operation, or hand manipulatable locking structure can be positioned at ball joints 342, 343 where a manual approach is being taken, or the cannulation system 340 is provided with a combination of automated and manual functionality and associated structure. A first arm 344 extending from a first ball joint 342 is connected to a second curved arm 345. The second curved arm 345 includes a track 346 along which a second ball joint 343 can be translated. Here, the second ball joint 343 holds the access kit 252. Again, servomotors can be configured at articulating joints to facilitate automated articulating movement of the first arm 344 and automated articulating and translational movement of the access kit 252 relative to the track 346 of the second arm 345. A servomotor (not shown) is also configurable to move the access kit 252 generally perpendicular to the ball joint 343 or curved arm 345. System directed coordination of the various moving parts of the cannulation apparatus 340 accomplishes placing the access kit 252 into position above the targeted insertion point and then the access kit 252 is advanced as desired within a graft or fistula as directed by the system. The system provides such directed coordination whether the cannulation system 340 is configured to be automated or manually controlled.

As shown in FIG. 16B, the cannulation system 340 can be mounted on a base 348 that is configured to engage the body part associated with the targeted insertion site. In one specific approach, the base 348 can define a stabilizer or distension element that can be configured to move to stretch, extend or stabilize the insertion site and also the graft or fistula so that it does not roll or otherwise move during cannulation. Such movement and application of stabilizing pressure by the base 348 can be automated by the system or can be manually controlled by the operator. The base 348 can be completely passive or can embody active arms or stays 349 that move together, apart, downwardly or at various angles to match needle insertion trajectory and to also apply even or uneven pressure as is deemed necessary or directed by the system. In this way, stability of the insertion site is provided while cannulating. Sensors (not shown) can also be provided so that force feedback is communication to the cannulation system and to the operator directly or through the system.

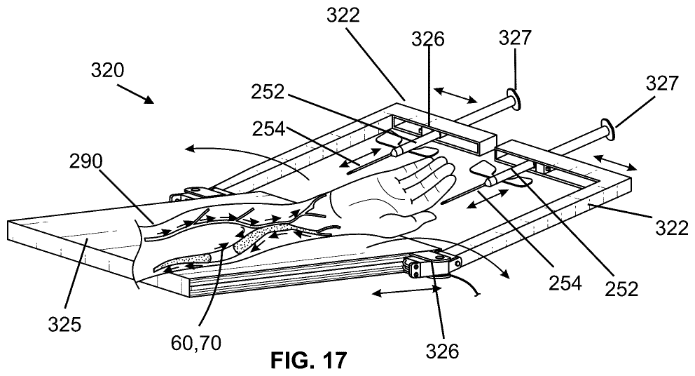

Turning now to FIGS. 17-19, the cannulation system 320 of FIG. 14 is shown in use to insert needles 254 of access kits 252 into targeted vasculature. Such a use can be for providing vascular access for hemodialysis. Moreover, certain of the functionality can be servomotor controlled by the system or otherwise performed manually as described.

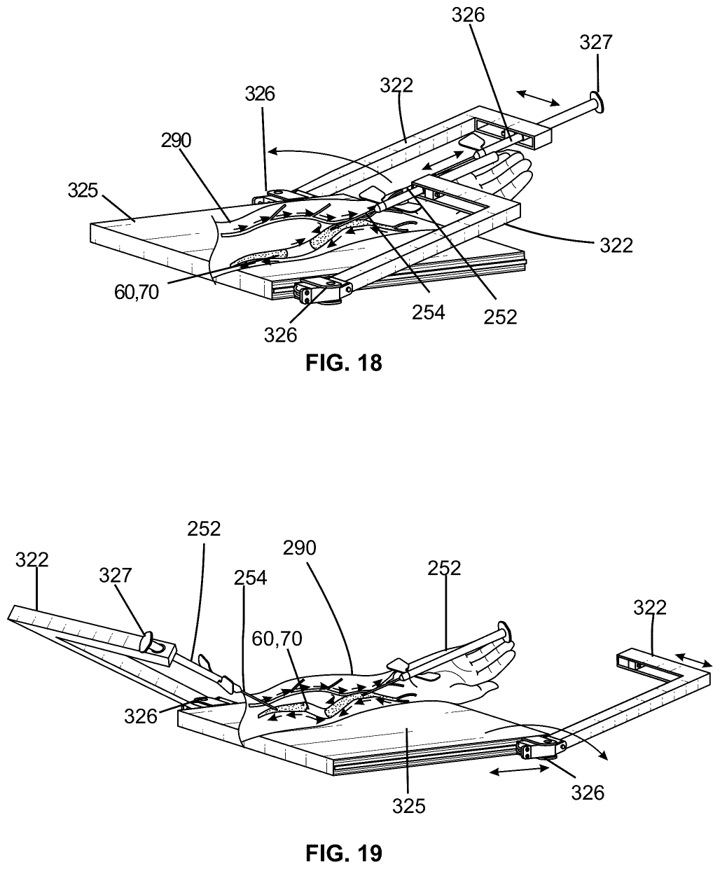

Once the positional relationship of implanted fiducials is established relative to a vessel or graft targeted for cannulation and a terminal depth of needle insertion has been established and stored within or communicated to the system, the body part or arm 290 is placed on the bed of the cannulation system 320. As stated, the arm 290 can be restrained (not shown) to the bed 325 so that a precise and repeatable orientation of the arm 290 to the bed 325 is provided. With the arm 290 positioned on the bed 325 as required and dictated, the system recognizes the patient and knows the path the needle is to take to accomplish cannulation. In one approach, a first L-shaped frame 322 carrying an access kit 252 is moved along the bed 325 and the access kit 252 is moved relative to the L-shaped frame 322 to position a needle 254 of an access kit 252 within a graft or vessel 60, 70 (FIG. 18). Depth of movement of the access kit 252 is controlled by the system 320 and can be limited by the stopper 327 attached to the access kit 252.

In an alternative embodiment, once cannulation has been achieved, the system can confirm successful cannulation. The system can prompt the patient or healthcare professional as to whether the cannulation has been successful and potentially sound an alarm if the cannulation has not been successful. The above-described sensor package can then be employed to continue to look at the cannulation to confirm that all is fine, including providing a visual to confirm the presence and location of the needle. Again here, one or more acoustic sensors can provide sound information regarding flow and IR can provide visual flow information, and a pressure sensor or visual sensor at a back of a needle or tubing can be provided to look for back flash and pulsing blood. A pull back of a needle plunger can be automated or manual.

The now inserted access kit 252 can be disengaged from the L-shaped frame 322 and connected to a tube running to a hemodialysis system (See for example FIG. 1). Next (FIG. 19), the second L-shaped frame 322 is manipulated to position the needle 254 of the second access kit 252 as required and dictated by the system 250 and within the targeted graft or vessel 60,70. Such action may require the L-shaped frame 322 to both be rotated with respect to the bed 325 and moved along the bed 325, and the access kit 252 to move relative to the L-shaped frame 322. After the needle 254 is placed as desired within the vessel or graft 60, 70, and proper cannulation is confirmed, this second access kit 252 can then also be disengaged from the L-shaped frame 322 and connected via a tube to a hemodialysis system. Dialysis can then commence as previously prescribed or directed by a physician.

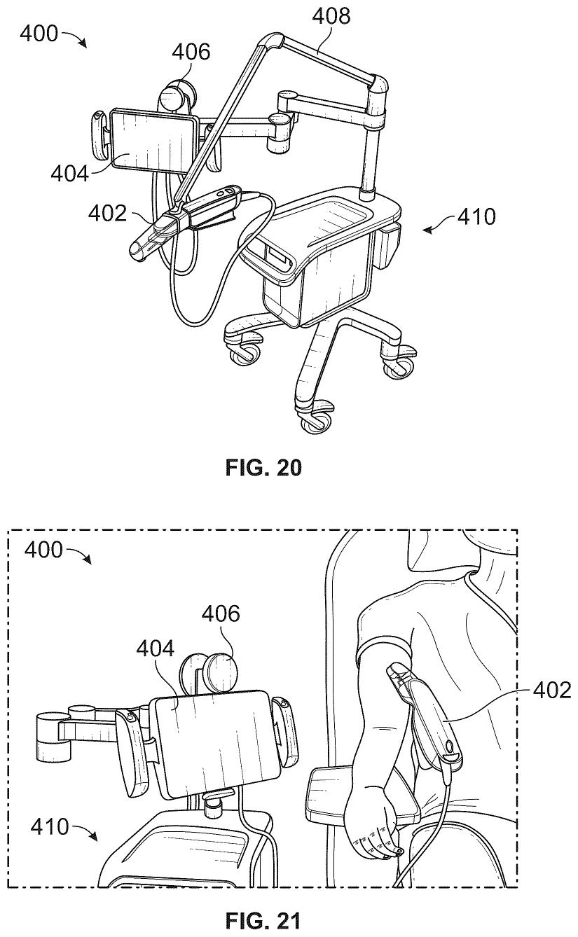

With reference to FIGS. 20-21, in an alternative embodiment there can be provided a cannulation system 400 including a handheld needle insertion assembly 402, whereby the components required to align a needle trajectory with a desired path is accomplished. The cannulation system 400 can include both mechanical and electrical components required to manipulate needle trajectory as well as sensing capabilities that assists in the fine positioning of a needle during insertion. In one particular aspect, the handheld needle assertion assembly 402 is configured to facilitate effectively and efficiently reaching challenging anatomy due to its size and minimal degrees of freedom.

The cannulation system 400 can further include a user interface 404 that allows a technician to interact with the handheld needle insertion assembly 402, a monitoring and localization probe 406 that is used to collect information about the target anatomy such as position, blood flow information, etc., and an articulating mounting arm 408 designed to stabilize the handheld needle insertion assembly 402. A cart 410 can be further provided and configured to contain a processing computer and control system designed to coordinate the actions of the various components of the cannulation system 400. The cart 410 may also contain the components required to provide power and data transmission to the rest of the system 400.

In one aspect, the cannulation system 400 can embody a handheld robot or needle insertion assembly 402 mounted on a passive arm 408 that is capable of being positioned and locked in place by the user or technician. The handheld robot or needle insertion assembly 402 that delivers the needle is designed to be small and can contain functionality and structure providing the degrees of freedom required to position and align a needle during insertion, as well as insert the needle into a target vessel. The handheld robot or needle insertion assembly 402 can include light or sound-based sensing (such as near infrared or ultrasound) on-board, intended to provide fine positioning information and assist in the needle placement. The monitoring and localization probe 406 probe can be configured to cooperate with the handheld robot or needle insertion assembly 402 and provide acoustic or light-based information that can monitor blood flow, pulse, etc., or also be used to locate a vessel by means of near infrared light and related processing capabilities. As stated, the cart 410 can further include processing capabilities as well as provide power and data processing/communication capabilities.

Referring specifically to FIG. 21, the handheld needle insertion assembly 402 is a small, portable device and can contain the mechanisms required to position, align, and insert a needle along a determined or desired path or trajectory. The handheld robot or needle insertion assembly 402 is electrically connected to power and data processing units, and can be connected to a support or mounting arm 408 or be handled independently of the support or mounting arm 408. The monitoring and localization probe 406 provides information to coordinate the motion of a needle as well as provides data about the target anatomy. In one embodiment, the handheld needle insertion assembly 402 also can contain a localization probe used to help with fine positioning and trajectory correction. Light based or acoustic signals can be employed to generate the information needed for monitoring or localization.

Thus, a system and method for accessing vessels in a consistent way without a need for skilled personnel is presented. Such a system can include, in one embodiment, implantable fiducials that are one or more of passive, active or activatable. The system includes a detection scheme for locating the fiducials in three-dimensional space and functionality designed to co-register such three-dimensional locations to a previously stored three-dimensional data set containing relative location information concerning target vessels. The stored data is retrieved and co-registered to three dimensional locations of prior access sessions and sites are re-used or sites of newly desired access points are determined. Other approaches employing sensors without fiducials can also be utilized to locate the cannulation site. Next, the access apparatus and system are guided to place an access kit into the target vessel with the desired orientation and depth and location.

As stated, in each or one or more of the disclosed embodiments and approaches, the patient can move the components of the system and advance the needles themselves, taking into account their own sensation, pain, or comfort, where the system ensures that the needle does not take an incorrect path or be inserted too deeply beyond a defined and desired terminal point. Additionally, in each or one or more of the disclosed embodiments and approaches, the system can automatically position, aim and advance a needle in a cannulation procedure. Further, in one or more of the disclosed embodiments and approaches, the patient and the system can work together to varying degrees and in various ways such as where the patient lets the system position the needles automatically but wants to push the needles into vasculature access site, or where the patient moves the structure holding the needles and lets the system push the needle into vasculature access site either when the system is ready or when the patient indicates they are ready and signals that cannulation should be performed.

In alternative embodiments, real time involvement of remotely located health professionals can be provided so that cannulation can be remotely visualized and guided, or the health professional can provide remote advice or information concerning a cannulation procedure. Telehealth video connection for real-time guidance of the system, troubleshooting, and recommendation on insertion path can be included via internet connectivity. In another alternative embodiment, augmented reality systems also can be incorporated into the system to both gather patient data as well as display real-time guidance for the cannulation needles or positioning of other components of an access apparatus.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the present disclosure.

That which is claimed is:

1. A method for positioning a needle into a vascular access site of a patient, comprising:
providing a needle insertion device and a needle retaining fixture attached to the needle insertion device;
guiding movement of the needle through an acceptable access path and preventing movement of the needle through non-acceptable paths or insertion of the needle beyond a defined terminal point; and
providing a health assessment of a fistula or graft prior to the needle insertion.

2. The method of claim 1, wherein the patient is scanned on a periodic basis to develop a map used to create the acceptable access path to be taken by the needle.

3. The method of claim 1, further comprising preventing the needle insertion should an obstruction be detected.

4. The method of claim 1, further comprising alerting a patient or healthcare provider should an obstruction be detected.

5. The method of claim 1, wherein the health assessment of a target insertion site involves one or more of recognizing vibrations, bruit or sounds associated with flow or obstructions.

6. The method of claim 1, further providing a real time assessment of the insertion procedure.

7. The method of claim 1, further comprising readjusting trajectory or depth of the needle insertion should the patient move before or between cannulations.

8. The method of claim 1, further comprising providing a handheld needle assertion assembly.

9. The method of claim 8, further comprising providing a user interface that allows a user to interact with the handheld assertion assembly, a monitoring and localization probe that is used to collect information about the target anatomy such as position or blood flow information.

10. The method of claim 9, further comprising providing a cart configured to contain a processing computer and control system designed to coordinate the actions of the various components of the cannulation system and including components required to provide power and data transmission to the rest of the system.

11. The method of claim 8, further comprising providing an articulating mounting arm designed to stabilize the handheld needle insertion assembly.
